SEQUENCE LISTING (15 sequences)

SEQ ID NO: 1 - DNA, 2082 bp, Arabidopsis thaliana
  CDS (1)...(2082): gene for ORE9
atggcttccactactctctccgacctccctgacgtcatcttatccaccatttcctctctcgtatccgattcccgagctcgcaactctctctccctcgtctctcacaaattcctcgctctcgaacgatccactcgctctcacctcactatccgtggcaacgctcgtgatctctccctcgtccccgactgtttccgatcaatctcacatctcgatctctctttcctctccccatggggtcacactcttctcgcttctctcccaatcgatcaccagaaccttctcgctctccgtctcaaattctgtttccctttcgtcgagtctctaaacgtctacacacgatctccgagctctctcgagcttctacttcctcaatggccgagaattcgccacatcaagctcctccgatggcatcaacgagcttctcagatccctaccggtggcgattttgttcctatttttgaacactgtggtggtttccttgagtctttagatctctccaacttctatcactggactgaagacttacctcctgtgcttctccgctatgctgacgtggcggcgaggcttacacggttagatctcttgacggcgtcgttcaccgagggatacaaatcaagcgaaatcgttagtatcaccaaatcttgccctaatttgaagacttttcgtgtagcttgtacgtttgatccgagatactttgaattcgtcggagacgagactctctccgccgtagctaccagttcccctaagttaacgcttctacacatggtggacacagcttcgttggcgaatcctagagctattccaggtacggaagctggagattcagctgtcacggcggggacgctaattgaagttttctcaggtttaccgaatctagaggagctggttcttgacgtaggaaaggatgtgaagcatagtggtgtagctttagaggcattgaattctaaatgcaagaagttaagagtattgaagctaggacagttccaaggtgtttgctctgctacagaatggaggaggctcgacggtgtggctttatgtggaggattgcagtcgttgtcgattaagaattccggcgatttgactgatatgggtttggtggctatagggagaggatgttgtaagttgactacgtttgagattcaagggtgtgagaatgtaacagtggatggactaagaacaatggttagtcttcggagtaagactttgactgatgtgagaatctcttgctgcaagaatcttgacacagctgcttctttaaaggcaattgagccgatttgtgatcggatcaagagactgcatatagactgtgtgtggtctggttcagaggacgaggaggtagaaggaagagtggaaactagtgaggctgaccacgaagaggaggatgatggttacgagaggagccagaagaggtgcaagtattcattcgaggaagaacactgctcaactagtgatgtgaatggattctgttctgaagatagagtatgggagaaactggagtatctatctttatggatcaatgttggagaatttttgacgccattacctatgacaggactagatgactgtccgaatttggaagagattaggatcaagatagaaggagattgcagaggtaaacgcaggccagccgagccagagtttgggttaagttgtctcgctctctacccaaagctctcaaagatgcagttagattgcggggacacaatcggtttcgcactgaccgcaccgccaatgcagatggatttgagtttatgggaaagattcttcttgaccggaattggaagcttgagcttgagcgagcttgattattggccaccacaggatagagatgttaaccagaggagtctctcgcttcctggagcaggtctgttacaagagtgcctgactttgaggaagctgttcatccatggaacagctcatgagcatttcatgaactttttgttgagaatcccaaacttaagggatgtacagcttagagcagactattatccggcgccggagaacgatatgagcacagagatgagagttggttcgtgtagccgattcgaggaccaattgaacagccgcaacatcattgactga

SEQ ID NO: 2 - protein, 693 aa, Arabidopsis thaliana
MASTTLSDLPDVILSTISSLVSDSRARNSLSLVSHKFLALERSTRSHLTIRGNARDLSLVPDCFRSISHLDLSFLSPWGHTLLASLPIDHQNLLALRLKFCFPFVESLNVYTRSPSSLELLLPQWPRIRHIKLLRWHQRASQIPTGGDFVPIFEHCGGFLESLDLSNFYHWTEDLPPVLLRYADVAARLTRLDLLTASFTEGYKSSEIVSITKSCPNLKTFRVACTFDPRYFEFVGDETLSAVATSSPKLTLLHMVDTASLANPRAIPGTEAGDSAVTAGTLIEVFSGLPNLEELVLDVGKDVKHSGVALEALNSKCKKLRVLKLGQFQGVCSATEWRRLDGVALCGGLQSLSIKNSGDLTDMGLVAIGRGCCKLTTFEIQGCENVTVDGLRTMVSLRSKTLTDVRISCCKNLDTAASLKAIEPICDRIKRLHIDCVWSGSEDEEVEGRVETSEADHEEEDDGYERSQKRCKYSFEEEHCSTSDVNGFCSEDRVWEKLEYLSLWINVGEFLTPLPMTGLDDCPNLEEIRIKIEGDCRGKRRPAEPEFGLSCLALYPKLSKMQLDCGDTIGFALTAPPMQMDLSLWERFFLTGIGSLSLSELDYWPPQDRDVNQRSLSLPGAGLLQECLTLRKLFIHGTAHEHFMNFLLRIPNLRDVQLRADYYPAPENDMSTEMRVGSCSRFEDQLNSRNIID

SEQ ID NO: 3 - protein, 326 aa, Arabidopsis thaliana
MASTTLSDLPDVILSTISSLVSDSRARNSLSLVSHKFLALERSTRSHLTIRGNARDLSLVPDCFRSISHLDLSFLSPWGHTLLASLPIDHQNLLALRLKFCFPFVESLNVYTRSPSSLELLLPQWPRIRHIKLLRWHQRASQIPTGGDFVPIFEHCGGFLESLDLSNFYHWTEDLPPVLLRYADVAARLTRLDLLTASFTEGYKSSEIVSITKSCPNLKTFRVACTFDPRYFEFVGDETLSAVATSSPKLTLLHMVDTASLANPRAIPGTEAGDSAVTAGTLIEVFSGLPNLEELVLDVGKDVKHSGVALEALNSKCKKLRVLKLG

SEQ ID NO: 4 - DNA, 22 bp, Artificial Sequence
  5′ primer for PCR of CAPS marker F14N22.6
caatactagacgtcttaaatgg

SEQ ID NO: 5 - DNA, 22 bp, Artificial Sequence
  3′ primer for PCR of CAPS marker F14N22.6
catagataagctgtcgttaatc

SEQ ID NO: 6 - DNA, 22 bp, Artificial Sequence
  5′ primer for PCR of CAPS marker F14N22.13
gatgatgatcgtcattttatgg

SEQ ID NO: 7 - DNA, 22 bp, Artificial Sequence
  3′ primer for PCR of CAPS marker F14N22.13
gatttctattcgtgatcgaaag

SEQ ID NO: 8 - DNA, 20 bp, Artificial Sequence
  5′ primer for PCR of ORE9
attgagtttgtactccggat

SEQ ID NO: 9 - DNA, 19 bp, Artificial Sequence
  3′ primer for PCR of ORE9
tctaaatagtttgaaacgg

SEQ ID NO: 10 - DNA, 37 bp, Artificial Sequence
  5′ primer for PCR of ORE9(1-49) fragment
ctacgcggatcctaaccatggcttccactactctctc

SEQ ID NO: 11 - DNA, 31 bp, Artificial Sequence
  3′ primer for PCR of ORE9(1-49) fragment
gatgcctgcaggattgatcggaaacagtcgg

SEQ ID NO: 12 - DNA, 31 bp, Artificial Sequence
  5′ primer for PCR of ORE9(50-693) fragment
ctacgcggatcctaggcaacgctcgtgatct

SEQ ID NO: 13 - DNA, 31 bp, Artificial Sequence; identical to SEQ ID NO: 11
  3′ primer for PCR of ORE9(50-693) fragment

SEQ ID NO: 14 - DNA, 32 bp, Artificial Sequence
  5′ primer for PCR of gene encoding ASK1 fragment
ctacgcgaattctaaccatgtctgcgaagaag

SEQ ID NO: 15 - DNA, 31 bp, Artificial Sequence
  3′ primer for PCR of of gene encoding ASK1 fragment
gatgcccatggtcattcaaaagcccattggt

(19) United States
(12) Patent Application Publication
(10) Pub. No.: US 2004/0123344 A1
(43) Pub. Date: Jun. 24, 2004

(54) Novel gene encoding an f-box protein which regulates leaf longevity in arabidopsis thaliana and mutant gene thereof

(76) Inventors: Hong Gil Nam, Kyungbuk (KR); Hye Ryun Woo, Kyungbuk (KR)

Correspondence Address: BURNS DOANE SWECKER & MATHIS L L P, POST OFFICE BOX 1404, ALEXANDRIA, VA, 22313-1404, US

(21) Appl. No.: 10/450,672

(22) PCT Filed: Dec. 19, 2001
(86) PCT No.: PCT/KR01/02204

(30) Foreign Application Priority Data
Dec. 20, 2000 (KR) 2000/78972

(51) Int. Cl. A01H 1/00; C12N 15/82; C12Q 1/68; C07H 21/04; C07K 14/415

(52) U.S. Cl. 800/278; 435/6; 435/69.1; 435/320.1; 435/419; 530/370; 536/23.6

(57) ABSTRACT

The present invention relates to an ORE9 gene that regulates leaft longevity in Arabidopsis thaliana, an ore9 mutant gene that delays the leaf longevity by repressing the physiological and biochemical changes involved in leaf senescence, and the use of the genes. The ORE9 gene regulating leaf longevity and the ore9 mutant gene thereof according to the present invention can be used for practical purposes such as improvement of plant productivity and pre- and post-harvest storage and development of the bioprobe for investigating the leaf longevity-associated gene and repressor of leaf longevity in plants.

### TECHNICAL FIELD

The present invention relates to a leaf longevity regulatory gene ORE9 isolated from Arabidopsis thaliana, a mutant type gene ore9, a mutant type of the ORE9 gene, that extends the leaf longevity by repressing the physiological and biochemical changes involved in leaf senescence, and the use of the genes.

### BACKGROUND ART

The suppression of plant senescence is not only of great scientific importance in itself, but also of great industrial importance in terms of the productivity of crops or the improvement possibility of post-harvest storage efficiency. For this reason, genetic, molecular biological, physiological and biochemical studies have been actively conducted in an attempt to establish plant senescence phenomena. In particular, studies on identification and function of genes using longevity-extended mutants provide the first step not only to establishing signal transfer pathways that affects the senescence progress rate, but also to solving the practical problems, such as improvement in plant productivity and an increase in the pre- and post-harvest storage efficiency of fruits, etc.

Senescence is the final stage that plants undergo during their lifetime. The initiation of senescence can be said to be a rapid changeover point in development of plants. During such a period, cells undergo dramatic changes in metabolism and cellular structure. In such changes of plants, one of the typical visual phenomena is the color change in autumnal leaves, autumnal tints, which appear when chlorophylls are destroyed and other pigments are produced. The chlorophyll breakdown occurring during the period of the autumnal tints involves chloroplast breakdown, and a decrease in anabolic activities, such as photosynthesis and protein synthesis. On the other hand, in this period, numerous hydrolases are induced while catabolism such as nucleic acid breakdown or proteolysis is activated (Matile P. et al., In Crop Photosynthesis: Spatial and Temporal Determinant, Elsevier 413-440, 1992; Nooden L. D. et al., Senescence and aging in plant, Academic press, 1988; and Thiman K. V. et al., The senescence of leaves, CRC press, 85-115, 1980). However, the plant senescence is seen as a process of cellular degeneration, and at the same time, a genetic character which is actively acquired for adaptation to environment during the development process, including migration of nutrients from growth organs to genital organs at the winter season. Senescence consists of a series of continuous biochemical and physiological phenomena and leads to the death of cells, organs and whole individuals (Matile P. et al., In Crop Photosynthesis: Spatial and temporal Determinant, Elsevier 413-440, 1992; Nooden L. D. et al., Senescence and aging in Plant, Academic press, 1988; and Thiman K. V. et al., The senescence of leaves, CRC press, 85-115, 1980; and Thomas H. et al., Annu. Rev. Plant Physiol. 123:193-219, 1993). Senescence is attributed to the gene theory which states that senescence is caused by genes according to a destined program, and the error accumulation theory which states that senescence is caused by information transfer error repeatedly occurring in vivo or error accumulation in a process of protein synthesis. The gene theory, which states that senescence is destined by genes, has been persuasive as of late. Thus, cloning of genes involved in plant senescence, and identification of the function of such genes, can be highly important in the study and regulation of senescence process. However, in spite of such scientific and practical importance, plentiful particulars on plant senescence are not yet known. In studies related to the plant senescence, reports connected with phytohormones have been the main area of interest until now, and molecular biological studies have recently begun.

Plant growth hormones (cytokinin) are known as hormones capable of physiologically delaying senescence. For this reason, there have been studies conducted to delay senescence by regulation of cytokinin secretion through regulation of senescence-associated genes. However, there are problems in that other physiological actions are affected due to the influence of hormones. Recently, in an attempt to solve these problems, IPT genes were linked to a promoter of senescence-specific SAG12 genes so that the plant growth hormones were regulated at a certain senescence stage so as to delay the progress of senescence, thereby achieving an increase of more than 50% in productivity while causing little or no changes in the blooming time and the like (Gan S et al, Science 22:1986-1988, 1995). In addition, for the development of plants having delayed senescence, a method has been attempted, which inhibits synthesis of ethylene, a material playing an important role in senescence, or reduces the amount of ethylene in cells, mainly in ripened tomatoes (Klee et al., Plant Cell, 3(11):1187-93, 1991; Oeller et al., Science, 18:254(5030):437-9, 1991; and Picton et al., Plant Physiol 103(4): 1471-1472, 1993).

Molecular biological studies for the delay of senescence are mainly focused on the manipulation of the relevant genes that have activities associated with biochemical changes occurring in a process of senescence or are involved in the signal transduction system. In the case of tomatoes, there are reported methods that prevent the expression of genes involved in the degradation of cell walls using antisense DNA so that the softening of tomatoes is prevented, thereby improving the transport and storage properties of tomatoes (Giovannoni et al., Plant Cell 1(1):53-63, 1989). It was also reported that, where the expression of phospholipase D is impeded with the antisense DNA, senescence caused by phytohormones is delayed (Fan et al., Plant Cell 9(12):2183-96, 1997).

However, methods capable of directly controlling plant senescence can be obtained by molecular biological studies which analyze the genes whose expression changes with senescence, and genetic studies that isolate and analyze senescence-associated mutants.

According to existing reports, it is known that, in Arabidopsis thaliana, the expression of ethylene receptors is controlled in a ripening period of fruits or in the senescence process of flowers (Payton S. et al., Plant Mol. Biol., 31(6):1227-1231, 1996), and the expression of clp genes is controlled in a senescence process of leaves (Nakabayashi K. et al., Plant Cell Physiol. 40(5):504-514, 1999). Recently reported were the identification of genes involved in a senescence process of leaves, using the Arabidopsis thaliana mutant type (Oh S. A. et al., Plant Mol. Biol. 30(4):739-54, 1996), three gene loci associated related therewith (Oh S. A. et al., The Plant Journal, 12(3):527-535, 1997), and a promoter activity of sen1, a senescence-associated gene, (Oh S. A., et al., Journal of Plant Physiology 151:339-345, 1997). However, molecular biological studies on genes which directly regulate senescence and their functions are, as yet, insufficient.

Therefore, the present inventors have made an effort to find mutants involved with the extended leaf longevity in Arabidopsis thaliana having many genetic advantages and to identify genes involved in longevity extension in the mutants, and consequently have found a mutant having an average leaf longevity longer than the wild type by about 27% and identified the relevant gene in the mutant on the basis of the genetic mapping. As a result, the senescence-associated gene was found to be a gene which is located at loci of m429 to 4.8±0.5 cM, particularly a locus of BAC F14N22, 693 amino acids on a cDNA sequence. This gene was termed ORE9. Furthermore, it was found that ORE9 protein coded with this gene has a modified F-box motif and 18 leucine-rich repeats (LRRs), while having controlled bonding between proteins similar to other existing proteins containing an F-box, and in the case of ore9, a mutant type of the ORE9 gene, the longevity of Arabidopsis thaliana is highly extended. Based on these points, the present invention was achieved.

### DISCLOSURE OF THE INVENTION

The present invention provides an ORE9 gene involved in senescence regulation, and ORE9 protein expressed from the ORE9 gene. The gene ORE9 is identified from the mutant type Arabidopsis thaliana having significantly extended leaf longevity when compared to the wild type. Furthermore, the present invention provides a method for identifying the senescence-associated gene or a substance capable of inhibiting senescence, using the senescence regulatory gene or protein.

In addition, the present invention provides a mutant type gene ore9 whose translation is terminated early by substitution of C, a 979th base of the ORE9 gene, with T. Also, it provides an ore9 protein expressed from the ore9 gene. The mutant type gene ore9 exhibits the ability to extend the plant longevity, and a method of extending the plant longevity by transforming plants with this mutant type gene ore9 is also within the scope of the present invention.

As used herein, the term “ore” was defined by the present inventors in the sense “live long”, so as to mean genes involved in the regulation of plant longevity, or proteins or derivatives expressed therefrom. Specifically, “ORE9 gene” or “ORE9 protein” designates longevity regulatory gene or protein, respectively, identified in the present invention. The term “ore9 gene” or “ore9 protein” means a mutant type gene of Arabidopsis thaliana obtained by generation of point mutation on a nucleotide sequence of ORE9, or a mutant type protein expressed therefrom, respectively.

Hereinafter, the present invention will be described in detail.

In the present invention, to identify a gene involved in longevity regulation, mutants showing the character of extended longevity were first selected. For this, Arabidopsis thaliana, which is frequently used as a subject for genetic and molecular studies of plants, was used as a test plant. Arabidopsis thaliana has completely decoded small genomes of 130 to 140 Mbp size in five chromosomes, and extensive genetic and physical maps thereof exist. Also, this plant has a short longevity, and has advantages in that it produces many seeds and is easily cultivated and transformed. For these reasons, this plant is frequently used as a genetic model for studies of plants (http://www.arabidopsis.org).

In the present invention, in order to select mutants with extended leaf longevity using Arabidopsis thaliana as the subject, the plant was treated with ethylmethyl sulfonic acid (EMS) to induce mutation. Then, individuals exhibiting a slow yellowing rate in their leaves were selected from grown individuals, and examined for survival rates of leaves, chlorophyll contents, photosynthesis efficiencies and ion outflow rates, so as to verify their character of extended longevity. The selected mutant was termed “ore9 mutant”, and their character was compared with that of the wild type. As a result, the ore9 mutant exhibits average leaf longevity of 31.4 DAE (days after emergence), which indicates about 27.1% increase in average longevity, compared to the wild type exhibiting an average leaf life of about 24.7 DAE. Also, regarding the progress rate of senescence, in the wild type, 50% of the chlorophyll was lost, but in the ore9 mutant, the yellowing phenomenon did not started until 24 DAE. Meanwhile, a reduction in photosynthetic activity and membrane ion outflow, which are other measures of the progression of senescence, indicated that senescence was progressed more slowly in the ore9 mutant compared to the wild type.

Although the senescence of a leaf is generally seen as destined within genes, it is known that the start and progress of senescence can be altered by phytohormones, such as abscisic acid (ABA), methyl jasmonate (MeJA) and ethylene (Hensel et al., Plant Cell 5:553, 1993). Thus, in order to examine a change in leaf longevity in the ore9 mutant according to treatment with such phytohormones, the progression of senescence was measured by examining chlorophyll contents and photosynthetic activities after the ore9 mutant and the wild type were treated with ABA, MeJA and ethylene, respectively. As a result, it was verified that, in the wild type, both the chlorophyll content and the photosynthetic activity were significantly reduced with influence of the phytohormones, so that senescence was accelerated. However, in the ore9 mutant, the effect of the phytohormones was highly reduced. This suggests that the ore9 mutant can be extended in its longevity even if it is treated with the senescence-accelerating hormones.

Meanwhile, in an embodiment of the present invention, in order to verify that an extension in longevity of the ore9 mutant is affected not only at a physiological level but also at a molecular level, the expression aspects of various senescence-associated genes were examined by Northern blotting analysis. For example, anabolic activity, such as photosynthesis, and self-maintenance gene activity are increased with the growth of leaves and decreased with the senescence stage (Nam et al., Curr. Opin. Biotech. 8:200, 1997). In the case of the wild type, the expression of photosynthesis-associated genes, such as chlorophyll a/b binding protein and chloroplast ribosomal protein S17, and the expression of genes, such as ribulose biphosphate carboxylase small subunit, are reduced in proportion to the progress of senescence. However, in the ore9 mutant of the present invention, there was little or no change in the expression of these genes. On the contrary, the expression of various senescence-associated genes, such as SAG12, SEN4 and SEN5, in the wild type, was increased as senescence progresses. However, it was found that the expression of the senescence-associated genes in the ore9 mutant was not substantially increased as senescence progresses. This fact suggests that the longevity extending effect of the ore9 mutant is exhibited not only at the physiological level but also at the molecular level.

In order to identify genes involved in controlling plant longevity, the genetic mapping was used. As a result, it was found that ORE9 is located at m429 to 4.8±0.5 centi Morgan (cM) loci, particularly BAC F14N22 locus, of chromosome 2 of Arabidopsis thaliana, on the genetic mapping. Based on this result, two cleaved amplified polymorphic sequence (CAPS) markers located at 0.05 cM and 0.1 cM loci were constructed, and a 10 kb domain containing three open reading frames (ORFs) was identified. From this, a 4.5 kb fragment containing only the ORE9 gene was subcloned, and it was verified that the fragment is sufficient for the complementation of the ore9 mutant.

A nucleotide sequence of the ORE9 gene provided according to the present invention, and an amino acid sequence of the ORE9 protein, are represented by SEQ ID NO: 1 and SEQ ID NO: 2, respectively. In a nucleotide sequence of the ore9 gene, a mutant type of ORE9, cytosine (C) which is a 979th base of the nucleotide sequence represented by SEQ ID NO:1 is substituted with thymine (T). In the ore9 protein expressed from the mutant type gene ore9, translation is terminated early. Thus, the ore9 protein has an amino acid sequence of 8 LRRs, which is shorter than the normal ORE9 protein having 18 LRRs. The amino acid sequence of the ore9 protein was represented by SEQ ID NO: 3.

The results of genomic DNA blot analysis revealed that the ORE9 gene is present in a single copy number within the genomes of Arabidopsis thaliana, and consists of 6 exons when comparing cDNA with gDNA. The ORE9 protein expressed from this gene consists of 693 amino acids. Also, it contains an N-terminal F-box motif, and has 18 LRRs. The identification of the amino acid sequence using a database indicated that the amino acid sequence exhibits 48.4% homology with Arabidopsis thaliana TIR1 involved in auxin response, 46.6% and 37.8% homologies with human CUL1 and FBL2 respectively, and 47.8% homology with yeast CDC4, and thus is homologous with F-box proteins containing LRRs.

The F-box motif is a hydrophobic sequence with a great degree of denaturation, and is a domain consisting of 40 amino acids. It is found in proteins which serve to collect substrates to a core of an ubiquitine ligase complex for ubiquitination and proteolysis (Craig et al., Prog. Biophys. Mol. Biol. 72:299, 1999). Specifically, the F-box proteins interact with Skp1 and Cdc53 proteins in ubiquitine-proteosome pathways, thereby forming an E3 ubiquitine ligase complex referred to as SCF (Skp1-Cdc53-F-box). Such F-box proteins are commonly found in the regulatory proteins of vertebrates and yeast, such as yeast Cdc4 and Grr1, human Skp2 and CUL1-pseudo proteins, etc. The F-box proteins were recently found in plants, and it was reported that these proteins also have an effect on the regulation of floral organ identity (UFO), JA-regulated defense (Coll), auxin response (TIR1) and the regulation of circadian clock (ZTL and FKF1) (see Xin et al., Science 280:1091, 1998; Ruegger et al., Genes dev. 12:198; 1998; Samach et al., Plant J. 20:433, 1999; Sommers et al., Cell 101:319, 2000; and Nelson et al., Cell 101:331, 2000).

In order to verify that the ORE9 protein, which shows sequence homology with various F-box proteins as described above, practically exhibits F-box protein activity, examination on whether the ORE9 protein can interact with ASK1 and ASK2, that are proteins analogous to Skp1 binding with the F-box proteins was carried out by the yeast two-hybrid assay. Plasmids, by which a fragment (1-49 a. a.) containing only an F-box region of ORE9, and a fragment (50-693 a. a.) from which the F-box portion was removed, are expressed in the form of a fusion protein with DNA binding domain of GAL4 (GAL4-BD), respectively, and plasmids by which ASK1 and ASK2 are expressed in the form of a fusion protein with a transcription activation domain of GAL4 (GAL4-AD), respectively, were constructed. Then, yeast was transformed with different combination pairs of the vectors, and cultured. Results indicated that only yeasts, which contain ASK1 plasmid and ORE9 containing the F-box region, are grown in a histidine-deficient medium, and exhibit β-galactosidase activity. On the other hand, it was found that the ORE9 derivatives from which the F-box region was removed, failed to bind to ASK1. Also, it was found that ASK2 and ORE9 bound to each other. This result suggests that there is specificity in binding between the F-box region of ORE9 and ASK proteins. As a result, it is concluded that the F-box region of ORE9 is a necessary for binding of ORE9 with ASK1, and ORE9 can perform similar functions to general F-box proteins.

Meanwhile, binding of the ORE9 protein to ASK1 was also verified by in vitro experiments. In vitro binding of radioactivity-labeled ORE9 and derivatives thereof with GST-ASK1 fusion protein shows that the ore9 mutant protein and also the ORE9 derivatives, containing the F-box region, were co-precipitated with GSK-ASK1. This suggests that these proteins directly bind to ASK1.

A mechanism by which the ORE9 protein has an effect on the leaf longevity of Arabidopsis thaliana can include the following two possibilities. One possibility is that the ORE9 protein acts as a negative regulator in the initiation of leaf senescence so that it serves to collect a transcriptional repressor which inhibits genes required for the initiation of senescence. Another possibility is that ORE9 acts as a receptor required for the selective degradation of self-regulatory proteins. In other words, as the F-box proteins play an important role in the protein degradation process via the ubiquitine pathway, ORE9 plays an important role in binding between proteins, like other F-box proteins. Thus, it can be said that ORE9 exhibits senescence phenomenon by the degradation of proteins via the ubiquitine pathway. In this case, it can be said that the reason for the expression of longevity extending character in the ore9 mutant is because C-terminal WD repeats or LRRs were removed from the ore9 proteins compared to the wild type ORE9, so that a binding force between the proteins was weakened.

However, the longevity regulatory or longevity extending effect achieved by the ORE9 gene and protein thereof, and ore9 gene and protein thereof according to the present invention, is not intended to be restricted by, or limited to the above theories, although its mechanism can be described by such theories.

Meanwhile, the ORE9 gene and ORE9 protein of the present invention are useful for investigating of senescence-associated genes or senescence inhibitory substances in plants. For example, genes having high sequence homology with the ORE9 gene can be investigated by comparing their nucleotide sequences with the ORE9 gene, or pseudo-genes can be investigated by performing hybridization reaction, using a fragment of the ORE9 gene as a probe, with cDNA produced using a template RNA or mRNA extracted from plants treated with senescence-associated substances. Furthermore, the genes of the present invention can be used to either investigate substances capable of directly binding to the genes of the present invention, as well as substances capable of inhibiting or activating the expression of the genes of the present invention, or to identify senescence inhibitory substances by analyzing the binding aspects of these substance to the ORE9 protein. Specifically, this analysis can be performed by various conventional methods including DNA chip method, polymerase chain reaction (PCR) and Northern blot analysis and Southern blot analysis, etc.

Moreover, when the ORE9 protein is used, an analysis identifying the expression aspect of the ORE9 protein can be carried out using a method selected from the group including an enzyme-linked immunosorbent assay (ELISA), a protein chip assay or a 2-D gel analysis, etc.

Meanwhile, the present invention provides a method for extending the longevity of plants by transforming the plants with the mutant type gene ore9. Regarding the method for producing the plants transformed with the mutant type gene, there may be plant transformation methods known in the art. For example, an Agrobacterium-mediated transformation method using a binary vector for plant transformation introduced with the mutant type gene ore9 can be used. In addition, when a vector not containing a T-DNA region is used, there may be electroporation, microparticle bombardment, polyethylene glycol-mediated uptake, etc.

Plants whose longevity can be extended by the method of the present invention includes dicotyledonous plants including a lettuce, a Chinese cabbage, a potato and a radish, and monocotyledonous plants including a rice plant, a barley, a banana and the like. When the method of the present invention is applied to edible greens or fruits, such as tomatoes, which have a thin pericarp and thus show rapid deterioration in quality caused by senescence, and plants whose leaf is mainly marketed, it effectively increases the storage efficiency of the plants.

### BRIEF DESCRIPTION OF THE DRAWINGS

FIG. 1 is a graph showing a survival rate of leaves depending on time, in Arabidopsis thaliana wild type and ore9, a longevity-extended mutant type thereof, in which the leaves are regarded as being dead when 80% of its total chlorophylls is lost, each of the populations consists of 100 independent leaves.

□: wild type

▪: ore9

FIG. 2a is a photograph showing changes of chlorophyll content depending on time, in Arabidopsis thaliana wild type, and ore9, a longevity-extended mutant type of the wild type.

FIG. 2b shows changes in photosynthetic activity depending on time, in which the photosynthetic activity is expressed in terms of photochemical efficiency (Fv/Fm) of PSII.

□: wildtype

▪: ore9

Fv: maximum variable fluorescence

Fm: maximum yield of fluorescence

FIG. 2c is a graph showing changes in outflow of membrane ions depending on time, in Arabidopsis thaliana wild type and ore9, a longevity-extended mutant type thereof, in which the membrane ion outflow is expressed as the ratio (percent) of an initial conductivity to a total conductivity.

FIG. 3 shows the results of Northern blot analysis on the expression patterns of senescence-associated genes (SAGs) and other photosynthesis-associated genes depending on time, in Arabidopsis thaliana wild type and ore9, a longevity-extended mutant type thereof.

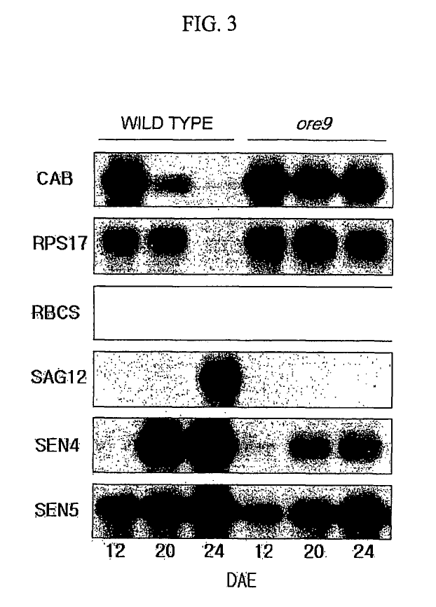

CAB: a represents chlorophyll a/b binding protein,

RPS17: a chloroplast ribosomal protein S17

RBCS: a ribulose biphosphate carboxylase small subunit

SEN4: a senescence-associated gene 4

SEN5: a senescence-associated gene 5

FIG. 4a is a graph expressing a change in the longevity of leaves, determined by photosynthesis efficiency, after treatment with abscisic acid (ABA), methyl jasmonate (MeJA) and ethylene, respectively, that are phytohormones having an effect on the initiation and progression of senescence.

Black bar: the leaves untreated with the phytohormones

White bar: the leaves treated with the phytohormones

FIG. 4b is a graph expressing a change in the longevity of leaves, determined by chlorophyll contents, after treatment with abscisic acid (ABA), methyl jasmonate (MeJA) and ethylene, respectively, which are phytohormones effecting the initiation and progression of senescence

Black bar: the leaves untreated with the phytohormones

White bar: the leaves treated with the phytohormones

FIG. 5a is a gene map showing a locus of an ORE9 gene in Arabidopsis thaliana genome.

Slant bar: a portion used in complementation assay of an ore9 mutant

FIG. 5b is a figure schematically showing the expected construction of ORE9 and ore9 proteins.

F: an F-box region

White box: LRRs

FIG. 6 shows the amino acid sequence homology and common sequences at an F-box region between ORE9 and proteins having an F-box motif.

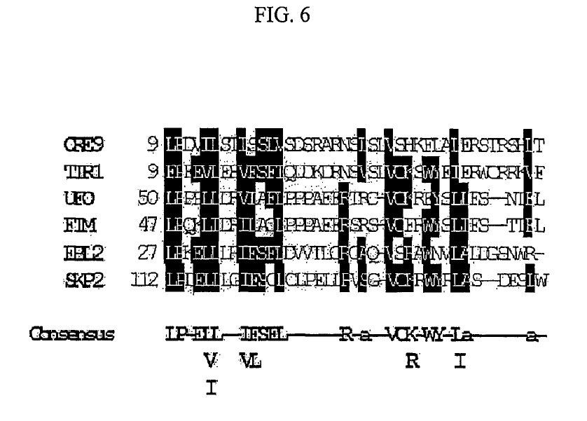

a: aliphatic amino acid residues

FIG. 7a is figure schematically showing the structure of ORE9, derivatives thereof, ASK1 and ASK proteins, used in the yeast two-hybrid assay.

Black box: GAL4-BD

Hatched box: GAL4-AD

FIG. 7b is a photograph showing results obtained after performing the yeast two-hybrid assay using ORE9 or derivatives thereof, and ASK1 or ASK2 protein.

The left upper portion: pairs and positions of plasmids used in the hybrid assay.

The right upper portion: results from cultivation in tryptophan and leucine-deficient plates.

The left lower portion: results from cultivation in tryptophan, leucine and histidine-deficient SD plates containing 2 mM of 3-amino-1,2,4-triazole (3-AT)

The right lower portion: β-galactosidase activities of transformants

FIG. 8 is a gel photograph showing the results of in vitro binding assay performed using ORE9 or derivatives thereof, and GSK or GSK-ASK1 fusion protein.

### BEST MODE FOR CARRYING OUT THE INVENTION

The present invention will hereinafter be described in further detail by embodiments. It should however be borne in mind that the present invention is not limited to or by the embodiments.

### EXAMPLE 1

### Selection of Longevity-Extended Mutants in Arabidopsis thaliana

About 40,000 of the seeds (M1) of Col-O, that is the wild type Arabidopsis thaliana were treated with a 0.33% ethylmethyl sulfonic acid (EMS) solution for 8 hours, and self-pollinated to obtain second-generation seeds (M2). The plants were then grown in a greenhouse at a controlled temperature of 23° C., and the yellowing of leaves caused by a reduction in chlorophylls according to age-dependent plant senescence was observed with the naked eye. Six individuals which had a slow yellowing rate compared to the wild type were selected. These selected mutants were named “oresara” which in the Korean language means “live long” (ore9, ore2, ore3, ore9, ore10 and ore11). From the results of biochemical analysis (analysis of chlorophyll contents, analysis of photochemical efficiencies, and analysis of RNase and peroxidase) for these mutants, these mutant plants were found to be practically functional stay-greens. The biochemical analyses are used as biomarkers for measuring leaf senescence. The longevity-extended mutants selected as described above and the wild type individuals to be used as a control group were grown in a growth room (Korea Instrument Inc.) under the conditions of 16 hours of light/8 hours dark at 22° C., prior to use in the subsequent tests. In the test, rosette leaf 3 and rosette leaf 4 were used.

### EXAMPLE 2

### Studies on Expression of Characters of Longevity-Extended Mutant ore9

In order to verify a longevity-extended character of the ore9 mutant, chlorophyll contents, photosynthetic activities and membrane ion outflows of leaves of the ore9 mutant were measured and compared with those of the wild type Arabidopsis thaliana.

2-1) Measurement of survival rates and chlorophyll contents of leaves

The leaf longevity was determined by measuring days after emergence (DAE), during which a 50% survival rate in leaves is maintained. The leaf longevity also was determined by the chlorophyll content of the leaves which was measured every four days after 12 DAE at which rosette leaf 3 and rosette leaf 4 are completely grown. When the leaves lost more than 80% of their total chlorophylls, they were determined to be dead. Sample groups used in this case were 100 independent leaves acquired from the respective individuals.

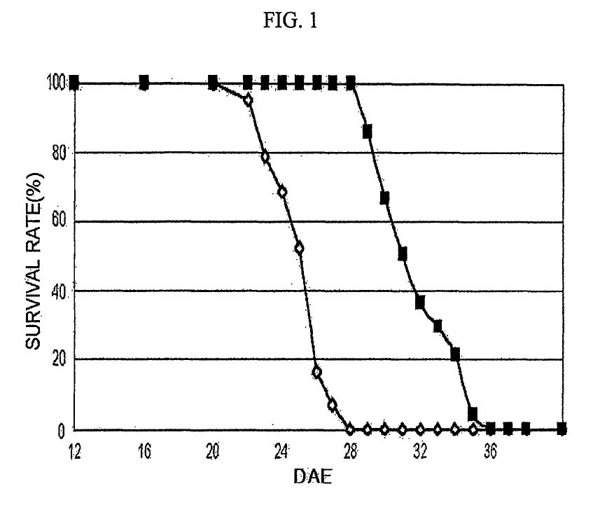

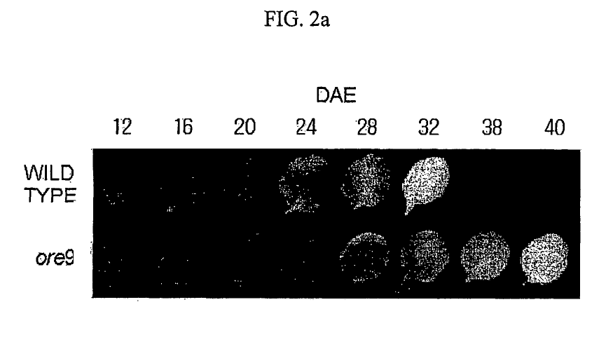

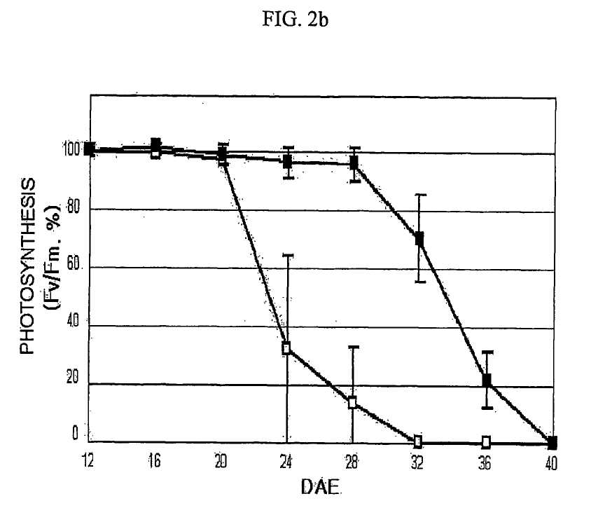

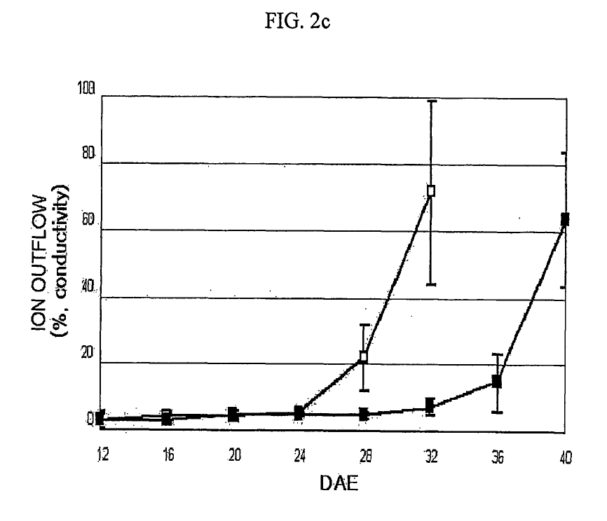

In order to measure the chlorophyll contents, the respective sample leaves were boiled in 95% ethanol at 80° C., thereby extracting chlorophylls. The chlorophyll contents were measured at absorbance of 648 nm and 664 run, and expressed as chlorophyll concentrations per fresh weight of leaves (Vermon et al., Anal. Chem. 32:1142-1150, 1960). As a result, as shown in FIG. 1, the average longevity of the wild type was about 24.7 DAE whereas the average longevity of the longevity-extended mutant ore9 was 31.4 DAE, which indicates a 27.1% increase in leaf longevity compared to the wild type. Also, as shown in FIG. 2a, the wild type lost about 50% of its chlorophylls at 24 DAE, but in the ore9 mutant, the yellowing phenomenon was just initiated at 24 DAE, and 50% of chlorophylls were maintained until 32 DAE.

2-2) Photosynthetic activities

In order to measure photosynthetic activities, a method of Oh S. A. et al., (Oh S. A. et al., Plant Mol. Biol. 30: 939, 1996) was used. First, the respective leaves were dark-treated for 15 minutes and measured for fluorescence of chlorophylls using a plant efficiency analyzer. The photosynthetic activity was expressed as photochemical efficiency of photosystemII (PSII) using a fluorescent property of chlorophylls. The photochemical efficiency was expressed as the ratio of maximum variable fluorescence (Fv) to maximum value of fluorescence (Fm) (Fv/Fm). As the ratio is increased, the photosynthesis efficiency improves.

Results indicated that a change in photosynthetic activities is similar to a change in chlorophyll contents. In other words, the photosynthetic activity in the wild type was rapidly decreased after 20 DAE, whereas the photosynthetic activity in the ore9 mutant was started to reduce after 28 DAE (see FIG. 2b).

2-3) Examination of membrane ion outflow

A membrane ion outflow was determined by measuring electrolytes flowing from leaves. Two leaves per individual of Arabidopsis thaliana were collected, immersed in 3 ml of 400 mM mannitol, lightly shaken for 3 hours at 22° C., and then measured for initial conductivity by means of conductivity meter SC-170. The sample was boiled for 10 minutes, and measured for total conductivity. Conductivity was expressed as the ratio (%) of the initial conductivity relative to the total conductivity.

In the wild type, the membrane ion outflow was rapidly increased after 24 DAE, but in the ore9 mutant, it was rapidly increased after 36 DAE (see FIG. 2c).

When considering the results as described above, it can be found that the ore9 mutant has phenotypes of prolonged leaf longevity than wild type. This longevity extending effect can be verified from the fact that the biochemical changes according to senescence, expressed as a reduction in chlorophyll contents, a reduction in photosynthetic activities, membrane ion outflow and the like, occur later than in the wild type.

### EXAMPLE 3

### Expression of Senescence-Associated Genes in ore9 Mutant

In order to verify that ore9 has any effect on the expression of senescence-associated genes (SAGs), the expression aspect of the respective SAG proteins according to the passage of time during a process of leaf development was identified by Northern blot analysis. The total RNA was isolated from leaves at 12, 20 and 24 DAE which are times of full growth, chlorophyll loss of less than 10%, and chlorophyll loss of more than 50%, respectively, on the basis of the wild type. 10 μg of RNA was loaded in every lane, and a full-length ORE9 gene was used as a probe.

It is known that anabolic activity, such as photosynthesis, and self-maintenance activity are increased with the growth of leaves and then decreased at the senescence stage (H. G. Nam, Curr. Opin. Biotech. 8:200, 1997). Results coincident to this report were also found in results of this embodiment. In the case of the wild type, the expression of photosynthesis-associated genes, such as chlorophyll a/b binding protein and chloroplast ribosomal protein S17, and the expression of genes, such as ribulose biphosphate carboxylase small subunit, were reduced as senescence progresses. However, in the ore9 mutant of the present invention, there were little or no changes in the expression of these genes. Meanwhile, in the wild type, the expression of various senescence-associated genes, such as SAG12, SEN4 and SEN5, was increased with the passage of time. However, it was verified that, in the ore9 mutant, the expression of the senescence-associated genes was not substantially increased in the same time. This fact suggests that the ore9 mutant delays the initiation of senescence at the physiological level and also at the molecular level, thereby extending the longevity of leaves.

### EXAMPLE 4

### Changes in Leaf Longevity of ore9 Mutant According to Treatment With Phytohormones

Although senescence of leaves is known as a genetic programmed process, the initiation and progress of senescence can be changed by phytohormones, such as abscisic acid (ABA), methyl jasmonate (MeJA) and ethylene, that are plant growth inhibitory substances (Hensel et al., Plant Cell 5:553, 1993; Weidhase et al., Physiol. Plant 69:161, 1987; and Zeevaart et al, Annu. Rev. Plant Physiol. Plant Mol. Biol. 39:439, 1998). Thus, in this experiment, a change in leaf longevity of the ore9 mutant according to treatment with phytohormones was examined by measuring changes in photosynthetic activity and chlorophyll content. Detached leaves were floated in 3 mM 2-[N-morpholino]-ethanesulfonic acid (MES) buffer, pH 5.8, containing 50 μM ABA or 50 μM MeJA, while continuously being exposed to light. Treatment with ethylene was carried out by cultivation in a glass box containing 4.5 μM ethylene gas. The treatments with phytohormones as described above were carried out for three days at 22° C. with continuous exposure to light. At this time, 12 independent leaves at 12 DAE were used as samples, and the chlorophyll content and the photosynthetic activity were measured in the same manner as in Example 2.

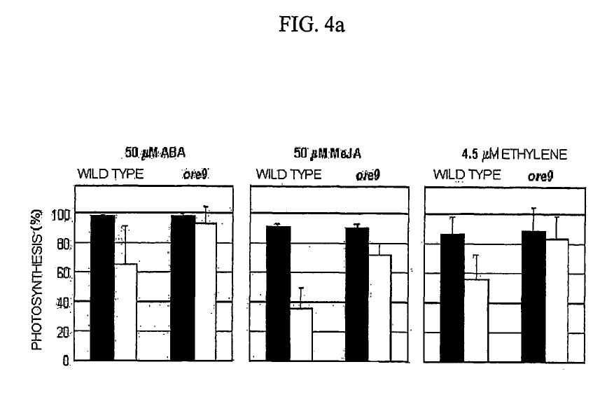

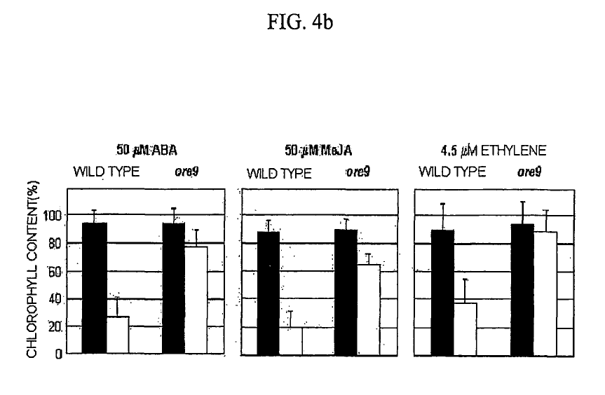

As a result, it was found that the photosynthetic activity of the wild type after treatment with ABA, MeJA and ethylene decreased to 65%, 38% and 54%, respectively, but the ore9 mutant maintained photosynthetic ability at 93%, 70% and 82%, respectively (see FIG. 4a). Furthermore, the reduction in the chlorophyll contents became blunt in the case of the ore9 mutant, similar to the photosynthetic activity (see FIG. 4b). These results suggest that the ore9 mutant has low susceptibility to the senescence-accelerating hormones and inhibits the progress of senescence caused by these hormones, so that it can extend the longevity of plants.

### EXAMPLE 5

### Cloning and Sequence Analysis of ORE9 Gene, Based on Genetic Mapping

In order to obtain accurate genetic information on ORE9, a gene map was constructed using cleaved amplified polymorphic sequence makers.

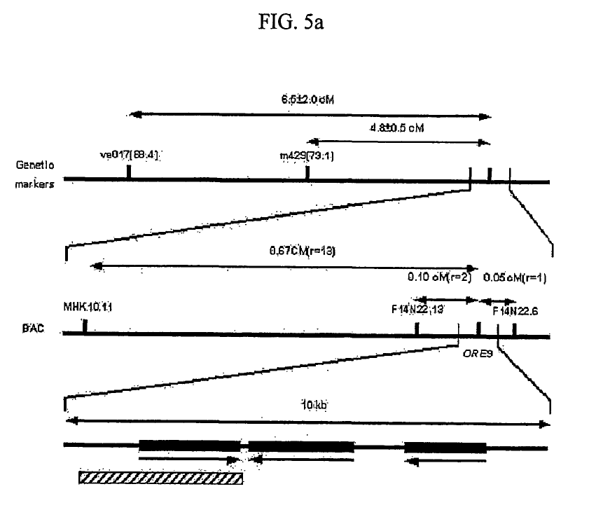

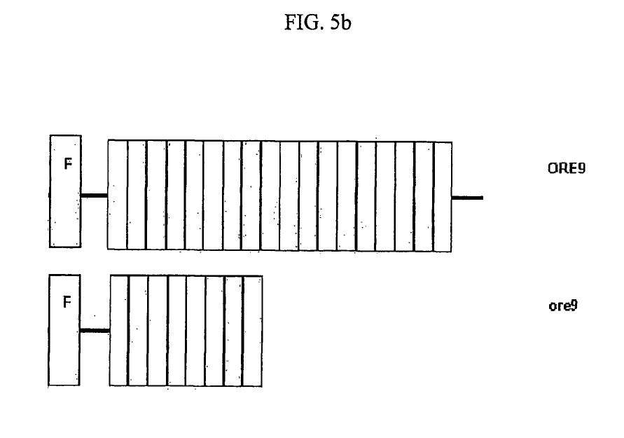

It could be found from the mapping that ORE9 is located at m429 to 4.8±0.5 centi Morgan (cM) loci, particularly BAC F14N22 locus on chromosome 2 (see FIG. 5a).

Two CAPS markers (F14N22.6 and F14N22.13) were constructed, which are located at 0.05 cM and 0.1 cM loci, respectively, at which one recombinant and two recombinants per 984 individuals can be obtained from ORE9, respectively. F14N22.6 of the CAPS markers is a product of a 1.2 kb size which was amplified by PCR using oligonucleotide having a nucleotide sequence represented by in SEQ ID NO: 4 and SEQ ID NO: 5, as a primer. Also, this marker contains two Dra I sites originated from Col, and three Dra I sites originated from Ler. F14N22.13 is a product of a 1.2 kb size which was amplified by PCR using oligonucleotide having a nucleotide sequence represented by SEQ ID NO: 6 and SEQ ID NO: 7, as a primer. Also, F14N22.13 contains one HinfI site originated from Col, and two HinfI sites originated from Ler. Mapping with these CAPS markers shows that a 10 kb region expected to contain the ORE9 gene contains three open reading frames (ORFs). Comparison of a nucleotide sequence of these three open reading frames with the ore9 mutant indicates that, in the ore9 mutant, a base C at one site of the open reading frames was substituted with T, so that translation is terminated early so as to produce protein shorter than the wild type (see FIG. 5b).

A 4.5 kb fragment containing only the ORE9 gene was subcloned into GEM T easy vectors (Promega, USA) by PCR, using oligonucleotides having a nucleotide sequence represented by SEQ ID NO: 8 and SEQ ID NO: 9, as a primer. Escherichia coli transformed with the resulting recombinant vector pGTE-ORE9 was deposited under the accession number KCTC 0881BP on Oct. 31, 2000 with the Korean Collection for Type Cultures (KCTC), Korean Research Institute of Bioscience and Biotechnology (KRIBB).

In order to examine the possibility that ORE9 can complement the longevity control in the ore9 mutant, the ORE9 gene-containing 4.5 kb fragment inserted into the recombinant vector was subcloned into a BamHI site of pCAMBIA1300 (MRC, USA), and the ore9 individuals were transformed with the subcloned vector. The transformed individuals were observed for antibiotic resistance and phenotype of T2 generation. Results indicate that the ORE9 gene-containing 4.5 kb fragment can complement the ore9 mutant, as shown in Table 1 below.

TABLE 1
Experiment for complementation of ore9 mutant by transgenes

| | Hygromycin | | | | | |
|---|---|---|---|---|---|---|
| | resistance | | | Phenotype | | |
| Genotype | Hyg^{R} | Hyg^{S} | X² | + | − | X² |
| Wild type | — | — | — | 25 | 0 | — |
| ore9 | — | — | — | 0 | 25 | — |
| ore9/ORE9-a | 215 | 68 | 0.143 | 56 | 19 | 0.004 |
| | | | (p > 0.5) | | | (p > 0.9) |
| ore9/ORE9-b | 231 | 73 | 0.158 | 53 | 18 | 0.005 |
| | | | (p > 0.5) | | | (p > 0.9) |
| ore9/ORE9-c | 276 | 14 | 1.001 | 67 | 6 | 0.483 |
| | | | (p > 0.1) | | | (p > 0.1) |

Note: Values of X² are for 3:1 or 5:1 (Hyg^{R}:Hyg^{S} or +:−) which is the phenotype ratio expected to be exhibited by descendants;
Hyg^{R}: Hygromycin resistance;
Hyg^{S}: Hygromycine susceptibility;
+: wild type;
−: longevity extended type;
ore9/ORE9-a, b, c: three independent T2 plants in which ore9 individuals are transformed with 4.5 kb fragments.

Meanwhile, the results of genomic DNA blot analysis indicated that ORE9 is present in genomes of Arabidopsis thaliana as a single copy number (data were not shown). Also, comparison of a cDNA sequence of ORE9 with the genomic sequence indicated that ORE9 consists of 6 exons. The cDNA sequence of ORE9 consists of 2082 bases encoding 693 amino acids and has a nucleotide sequence represented by SEQ ID NO: 1. ORE9 protein encoded with the ORE9 gene has a degenerated F-box motif and 18 incomplete LRRs (see FIG. 5b).

### EXAMPLE 6

### Identification of Functions of ORE9 as an F-Box Protein in SCF Complex

A polypeptide sequence analogized from the nucleotide sequence of the ORE9 gene identified in Example 5 was identified using databases. Results indicated that ORE9 protein is homologous with Arabidopsis thaliana TIR1 involved in auxin response (48.4%), and also with F-box proteins containing LRRs, such as human CUL1 (46.6%) and FBL2 (37.8%), and yeast CDC4 (47.8%). The F-box proteins interact with Skp1 and Cdc53 proteins in ubiquitine-proteosome pathways, thereby forming an E3 ubiquitine ligase complex referred to as SCF (Skp1-Cdc53-F-box) (Craig et al, Prog. Biophys. Mol. Biol. 72:299, 1999). Thus, in order to verify that the ORE9 protein functions as the F-box protein serving to form the SCF complex, examination on whether the ORE9 protein interact with Skp1-like proteins or not was carried out by yeast two-hybrid assay and in vitro binding assay. As the Skp1-like proteins, Arabidopsis SKP1 homolog 1 (ASK1) and Arabidopsis SKP1 homolog 2 (ASK2) were preferably used.

6-1) Construction of Expression Vector for Yeast Two-Hybrid Assay

As expression vectors of expressing GAL4 DNA binding domain (GAL4-BD) and ORE9 fusion protein, plasmids pGBT9-ORE9 (1-49) [1] and pGBT9-ORE9 (50-693) [2] were constructed. The plasmid pGBT9-ORE9 expresses a fragment of ORE9 containing N-terminal 1-49th amino acids corresponding to an F-box region of ORE9, and the plasmid pGBT9-ORE9 (50-693) expresses a fragment of ORE9 containing 50-693th amino acids from which the F-box region was removed.

First, the gene encoding the ORE9 (1-49) fragment was amplified by PCR using a primer represented by SEQ ID NO: 10 and SEQ ID NO: 11, and then inserted into BamHI and PstI restriction enzyme sites of pGBT9 (Clontech, USA) containing a 4-BD gene and a tryptophan auxotrophic selection marker gene (TRP1), thereby constructing the plasmid pGBT9-ORE9 (1-49). Similarly, the plasmid pGBT 9-ORE9 (50-693) was constructed, which expresses an ORE9 (50-693) fragment and a GAL4-BD fusion protein. In this case, oligonucletides represented by SEQ ID NO: 12 and SEQ ID NO: 13 were used as PCR primers to amplify the gene encoding the ORE9 (50-693) fragment.

Meanwhile, regarding ASK1(1-160) and ASK2(1-172) proteins used for identification of binding, pGAD424-ASK1(1-160) and pGAD424-ASK2(1-172) were constructed in such a manner that ASK1(1-160) and ASK2(1-172) are expressed in the form of a fusion protein with a GAL4-BD. pGTE-ASK1(1-160) containing an ASK1(1-160) gene was digested with BamHI and PstI, and then inserted into pGAD424 (Clontech, USA) containing a GAL4-AD gene and a leucine auxotrophic selection marker gene (LEU2), thereby constructing the plasmid pGAD424-ASK1(1-160)[3] expressing fusion protein of ASK1 and GAL4-4AD. Similarly, the plasmid pGAD424-ASK2(1-172)[4] was constructed by inserting a BamHI/PstI fragment of pGTE-ASK2(1-172) into pGAD424.

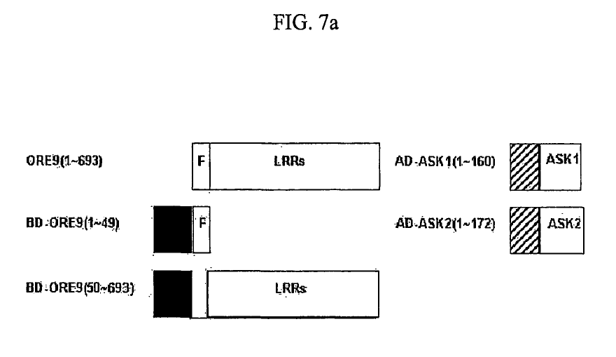

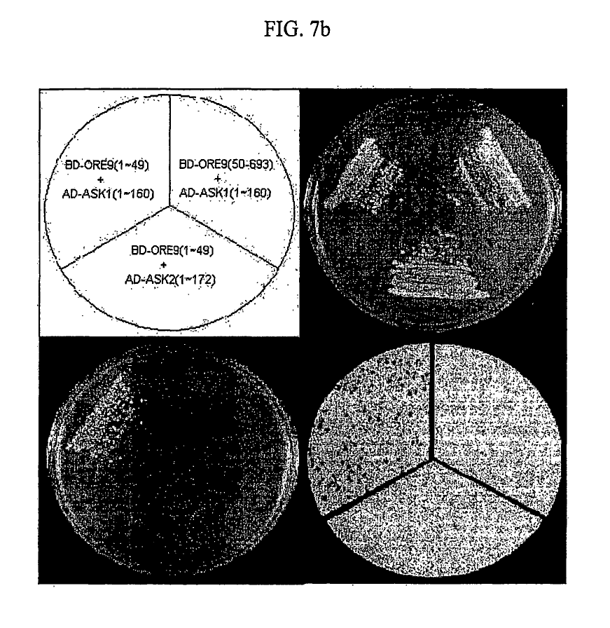

FIG. 7a schematically shows a construction of the fusion protein expressed from the plasmids constructed as described above.

6-2) Yeast Two-Hybrid Assay

Yeast strains, HF7c, were cultured in an YPD (yeast extract, peptone, dextrose) medium or a synthetic minimal medium (SD) containing 2% dextrose.

The HF7c yeast strains grown in the medium were transformed with different combinations of the vectors constructed in the above Example 6-1) ([1+3], [2+3] and [1+4]; see the left upper portion of FIG. 7b), respectively, by the lithium acetate method (Feilotter et al., 1994). The transformants were selected in a synthetic minimal medium containing 2% dextrose. The transformants were cultured in a SD medium from which tryptophan and leucine were removed (see the right upper portion of FIG. 7b), and in a SD medium from which tryptophan, leucine and histidine were removed and which contains 2 mM 3-amino-1,2,4-triazole (3-AT) (see the left lower portion of FIG. 7b). Results show that only yeasts transformed with the vector pGBT-ORE9(1-49)[1] containing the F-box region and the vector pGAD-ASK1(1-160)[3] expressing ASK1(1-160) are grown in the histidine deficient medium.

However, it was found that the ORE9 derivatives from which the F-box region was removed [ORE9(50-693)] fails to bind with ASK1 (see FIG. 7b). This fact suggests that the F-box region is a necessary and sufficient condition for binding of ORE9 with ASK1.

Meanwhile, the yeast two-hybrid assay revealed that ASK2 and ORE9 didn't bind to each other. This result suggests that there is specificity in binding between the F-box region of ORE9 and ASK proteins. However, the complete ORE9(1-693) did not exhibit positive signals for binding to ASK1 (data were not shown). This is believed to attribute to misfolding of the fusion protein or separation of proteins from nuclei, etc.

The transformants were grown in a synthesis minimal medium, and the grown yeast colonies were examined for β-galactosidase activities on a filter paper. The filter paper was left to stand in liquid nitrogen for 30 seconds, and cultured in a Z buffer (60 mM Na₂HPO₄, 40 mM NaH₂PO₄, 10 mM KCl, 1MM MgSO₄) containing 0.82 mM 5-bromo-4-chloro-3-indolyl-β-D-galactosidase (X-gal). The filter paper was maintained at 30° C., and observed for color change that indicates β-galactosidase activity.

Results revealed that only yeasts containing the ASK1(1-160) plasmid and the ORE9 [ORE9(1-49)] containing the F-box region, which exhibited the ability to grow in the histidine deficient medium, exhibit the β-galactosidase activity.

6-3) In vitro Binding Assay

In order to examine that ORE9 protein or derivatives thereof directly bind with ASK1, a GST-ASK1 fusion protein and a ³⁵S-labeled ORE9 protein produced by in vitro translation were subjected to in vitro binding assay. To obtain a glutathione S-transferase (GST) protein and a GST-ASK1 fusion protein, a vector pGEX (APBiotech, Co.) and a vector pGEX-ASK1 were constructed, which express the above proteins, respectively. Artificial sequences represented by SEQ ID NO: 14 and SEQ ID NO: 15 were used as PCR primers to amplify a gene encoding the ASK1 fragment. The resulting PCR product was digested with EcoRI and NcoI restriction enzymes, and inserted into the restriction enzyme sites of the pGEX vector. Escherichia coli BL21(DE3) pLysS was transformed with the resulting two vectors, and GST and GST-ASK1 fusion proteins were expressed. The GST and GST-ASK1 fusion proteins were purified by chromatography with glutathione-Sepharose 4B beads. The purified proteins was subjected to electrophoresis with SDS-polyacryamide gets, dyed with Coomassie blue, and measured for their amount. GST and GST-ASK1 fusion proteins of the same amount were added to glutathione beads, which had been previously washed with 10-fold volume of B buffer (20 mM chloride-phosphate, pH 7.6, 150 mM sodium chloride, 10% glycerol, 0.5% NP-40, 1 mM DTT) three times. Then, the resulting proteins were adsorbed 4° C. for one hour using a rotating mixer. The provided beads were washed with 1 ml B buffer three times, and then stored in a state where the ratio of slurries relative to B buffer is 50%.

Meanwhile, a radioactivity-labeled ORE9 (1-693), the mutant type ore9 and ORE9 derivatives (1-327 and 50-693) were prepared using the in vitro transcription/translation system (Promega, USA) and [³⁵S]-methionine (DuPont NEN, USA). The proteins were quantitatively analyzed with SDS-PAGE and BAS radioanalytic imaging system (Japan). Then, the respective ³⁵S-labeled translation products of the same amount were mixed with 60 μl GST-adsorbed beads or GST-ASK1 fusion protein-absorbed beads, contained in 1 ml GB buffer (final concentration: 20 mM Tris-HCl, pH 7.5, 0.15% NP-40, 150 mM NaCl, 1 mM EDTA). The resulting beads were cultured in a rotating mixer at 4° C. for two hours, and then washed with 1 ml GB buffer four times. The washed beads were added with 30 μl of 2×SDS sample solutions, boiled for three minutes and then isolated with SDS-PAGE. The resulting gels were dried to dryness, and then exposed to X-ray film.

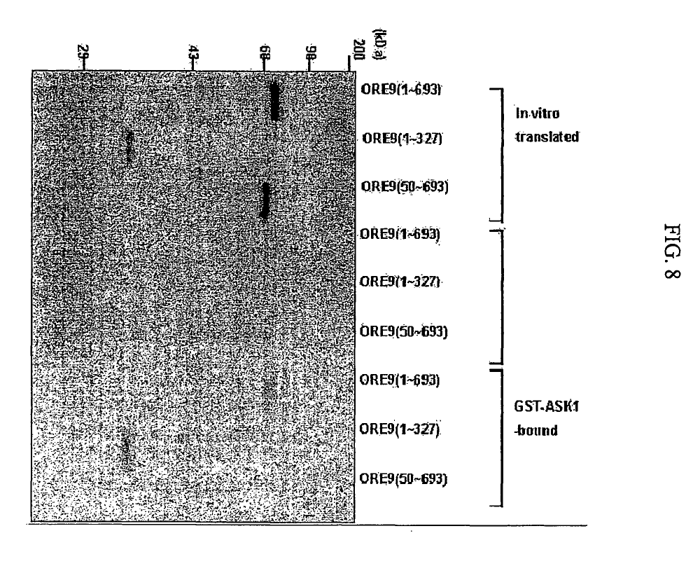

Results indicated that the complete ORE9 (1-693) directly bound to ASK1, unlike the yeast two-hybrid assay. Moreover, the mutant type ore9 and the part of ORE9 (1-327) containing the F-box region were co-precipitated with the GST-ASK1 fusion protein, but not co-precipitated with the GST protein, a negative control group (see FIG. 8). Meanwhile, it was found that ORE9 (50-693), from which the F-box region was removed, fails to bind with GST-ASK1. These results suggest that ORE9 directly binds with ASK1.

### INDUSTRIAL APPLICABILITY

The novel senescence regulatory gene ORE9 of the present invention and the ORE9 protein expressed therefrom, are useful for studies of senescence mechanisms, and for identification of senescence-associated genes or inhibitory substances, in plants. Furthermore, plants can be transformed with the ore9 gene, a mutant type of the ORE9 gene, so that the longevity of plants is extended, thereby achieving improvement in productivity and an increase in storage efficiency of the plants.

What is claimed is:

1. A protein ORE9 regulating longevity in plants, which has an amino acid sequence represented by SEQ ID NO: 2.

2. The protein ORE9 according to claim 1, in which the protein is isolated from Arabidopsis thaliana.

3. The protein ORE9 according to claim 1, which has an N-terminal F-box motif, and 18 leucine-rich repeats(LRRs).

4. A gene encoding the ORE9 protein of claim 1.

5. The gene according to claim 4, which has a nucleotide sequence represented by SEQ ID NO:1.

6. A recombinant vector, which contains the plant longevity-extending gene of claim 4.

7. The recombinant vector according to claim 6, which is pGTE-ORE9 (accession number: KCTC 0881BP) containing the ORE9 gene.

8. A mutant type gene ore9 extending longevity in plants, which has a nucleotide sequence in which cytosine (C), that is a 979th base in a nucleotide sequence represented by SEQ ID NO: 1, is substituted with thymine (T).

9. An ore9 protein which is expressed from the ore9 gene of claim 8, and has an F-box motif and 8 leucine-rich repeats.

10. A method for investigating a senescence regulatory gene or protein in plants by utilizing an ORE9 gene, an ORE9 protein, an ore9 gene, an ore9 protein, or fragments or derivatives thereof.

11. The method according to claim 10, which examines sequence homology of gene, hybridization reaction or protein binding reaction by methods including a DNA chip method, a protein chip method, a polymerase chain reaction (PCR), a Northern blot analysis, a Southern blot analysis, an enzyme-linked immunosorbent assay (ELISA) and a 2-D gel analysis.

12. A method for increasing productivity and storage efficiency of plants, which comprises transforming the plants with a mutant type gene of ORE9 gene, so that longevity of the plants is extended.

13. The method according to claim 12, in which the mutant type gene is an ore9 gene which has a nucleotide sequence in which cytosine (C), that is a 979th base in a nucleotide sequence represented by SEQ ID NO: 1, is substituted with thymine (T).

14. The method according to claim 12, in which the plants are selected from the group consisting of food crops including a rice plant, a wheat, a barley, a corn, a bean, a potato, an Indian bean, an oat and an Indian millet; vegetable crops including an Arabidopsis sp., a Chinese cabbage, a radish, a red pepper, a strawberry, a tomato, a watermelon, a cucumber, a cabbage, a melon, a pumpkin, a stone-leek, an onion and a carrot; special crops including a ginseng, a tobacco plant, a cotton plant, a sesame, a sugar cane, a sugar beet, a Perilla sp., a peanut and a rape; fruit trees including an apple tree, a pear tree, a jujube tree, a peach tree, a kiwi fruit tree, a grape tree, a citrus fruit tree, a persimmon tree, a plum tree, an apricot tree and a banana tree; flower crops including a rose, a gladiolus, a gerbera, a carnation, a chrysanthemum, a lily and a tulip; and fodder crops including a ryegrass, a red clover, an orchardgrass, an alfalfa, a tallfescue and a perennial ryegrass, etc.
